(19) United States
(12) Patent Application Publication
(10) Pub. No.: US 2006/0052857 A1
(43) Pub. Date: Mar. 9, 2006

(54) Pacemaker electrode array

(75) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(73) Assignee: Peter Osypka Stiftung, Grenzach-Wyhlen (DE)

(21) Appl. No.: 10/537,983

(22) PCT Filed: May 7, 2004
(86) PCT No.: PCT/EP04/04864

§ 371 Date: Jun. 8, 2005

(30) Foreign Application Priority Data
May 13, 2003 (DE) 103 21 337.6

(51) Int. Cl. A61N 1/05

(52) U.S. Cl. 607/119

(57) ABSTRACT

A pacemaker electrode array (1) which includes a pacemaker electrode (5) that acts upon the outside of a heart (2) and that is disposed with one pole (3), in the operational position, in the cardiac tissue and extends to an implanted pacemaker (4). The array includes a puncturing needle (7) and an insertion tube (8) for the puncturing needle to assist in installation. The inner diameter of the tube is sufficiently large enough for the pacemaker electrode (5) to fit into the insertion tube (8) once the puncture needle (7) is withdrawn and to be introducible into the cardiac tissue while inserted in the tube. The introduction tube (8) can be withdrawn in a known manner or can be removed laterally through a lateral opening.

### BACKGROUND

The invention relates to a cardiac pacemaker electrode arrangement with a cardiac pacemaker electrode, which, in the operational position, acts on the outside of a heart or acts on the heart from the outside and/or which is arranged or can be fixed with a pole in the heart tissue, which extends to an implantable cardiac pacemaker, and which has an electrode feed line, and with an anchor that can be fixed from outside of the heart in the operational position, and with at least one tool and/or aid for positioning and fixing the anchor.

Such electrode arrangements for myocardial stimulation of the heart are known from DE 2 219 044.9 and U.S. Pat. No. 4,355,642.

Here, epicardial screw electrodes are used as the anchor, for which the problem arises that they must be screwed into a fat-free area of the outside of the heart in order to achieve an adequate stimulus threshold. Such electrodes also require visual contact with the heart during their implantation. The necessary size of these electrodes and their anchoring can also lead to considerable problems primarily in a child's heart. If biventricular stimulation of the heart is necessary, these relatively large anchors of individual electrodes must be mounted on the left and on the right ventricle. Here, it is problematic primarily in finding a fat-free or low-fat region of the heart surface, which is thus a good electrical conductor and which is simultaneously arranged at the proper position of the heart for the stimulation. Furthermore, a correspondingly large space requirement is produced by the required, relatively large screw thread.

### SUMMARY

Therefore, the objective arises of creating an electrode arrangement of the above-noted type, in which the thorax must be barely opened, so that minimally invasive surgery on the heart is sufficient, but nevertheless the electrode can be fixed rather securely in the myocardium without having to take into account an outer fat layer on the heart. Simultaneously, the space requirement for attaching the electrode to the outside of the heart should be as small as possible, while simultaneously the stimulating line should be as good as possible.

To meet this objective, the cardiac pacemaker electrode arrangement defined above is characterized in that at least one hollow puncture needle and one insertion tube for this hollow puncture needle are provided as tools or aids and in that the inner cross section of the insertion tube is dimensioned so that the cardiac pacemaker electrode with its anchor provided on the distal end fits in the tube and can be moved in this tube.

Such a cardiac pacemaker electrode arrangement enables the anchor and thus the end of the electrode to be placed in the interior of the myocardium independent of any fat layer on the outside of the heart, so that good fixation and also the best-possible stimulating line are achieved. In this way, advantageously only a minimal opening in the thorax is sufficient to be able to anchor the electrode onto the heart, because advantageously a hollow puncture needle that can be used for preparing the anchoring can be inserted with an insertion tube to the corresponding position of the heart and then can be removed from this tube in a known way, after which the actual electrode can be inserted through this tube into the prepared opening in the heart and anchored.

Thus, after withdrawing the hollow puncture needle, the cardiac pacemaker electrode can be inserted into the heart tissue and into the channel formed in this tissue by the hollow puncture needle through the insertion tube leading from the outside into the heart tissue. Thus, in a space saving way with a small anchor, nevertheless an attachment in the myocardium can be implemented effectively, without a fat layer on the outside hindering this anchoring or even leading to an inadequate anchoring.

It is especially useful for a good anchoring and a good stimulating line if a screw thread, whose center axis forms the continuation of the longitudinal center axis of the cardiac pacemaker electrode, is arranged at the distal end of the cardiac pacemaker as an anchor; if the cardiac pacemaker electrode is flexible, such that it can be twisted; and if a channel is arranged in it for a piercing instrument used as the tool or as an additional tool and a profiled or shaped section, especially a flattened shape or recess, is arranged in the region of the screw electrode, which fits together with the working end of the piercing instrument or tool—for example, a screwdriver-like working end—with a positive fit in the direction of rotation.

Through such a configuration of the invention, it is possible to turn and twist the cardiac pacemaker electrode with its screw thread configured as an anchor opposite the screw direction and to fix it in this position with the help of the piercing instrument, then to insert it and finally to turn it within the myocardium into the released position and therefore also to screw in and anchor the screw thread into the myocardium. Then, in a typical way, the insertion tube can be removed, optionally with the removal from a longitudinal side, as known, for example, also for insertion sleeves according to DE 34 20 455 C1 in an analogous way.

One modification or further embodiment of the electrode arrangement according to the invention can arise in that at least one ring-like, screen-like, and/or barb-like projection is arranged as the anchor or as an additional anchor.

It is also possible, instead of a screw thread, to provide barb-like projections, which act as the anchor and which produce an automatic anchoring in the myocardium primarily after insertion through the insertion tube. It is further possible, in an electrode arrangement with an anchor formed as a screw thread, to use additional barb-like projections as one or more other anchors in order to improve the attachment in the myocardium even more.

Several anchors can also be provided in the axial direction in order to improve the attachment to the heart.

The electrode arrangement can be provided as a biventricular arrangement and a common feed line can have two electrodes that branch off and then run separately to the heart. Each electrode has at least one anchor for attachment to the heart. Here, the common feed line can lead from a control device or from the cardiac pacemaker for the electrodes as close as possible to the heart, so that the branched electrodes are correspondingly short and can be guided easily through the thorax to the heart in order to be able to transmit corresponding stimulation pulses to the heart in the operational position.

The anode of the electrode or the electrodes can be arranged outside of the heart at a distance to the cathode or to the pole located on the heart and, for a biventricular, branched electrode, in the region of the common feed line before the branching. Therefore, the cross section in the myocardial channel can be held as small as possible and the permanent load on the heart by the electrode arrangement anchored to it can be held to a minimum.

Primarily for the combination of individual or several of the previously described features and means, an electrode arrangement is produced, which can be anchored with a space-saving arrangement and with a good stimulating line and nevertheless with a permanent attachment on the outside of the heart in the myocardium, without requiring a large opening in the thorax for this procedure.

### BRIEF DESCRIPTION OF THE DRAWINGS

Embodiments of the invention are described in more detail below with reference to the drawing. Shown in partially schematized representation are:

FIG. 1 a cardiac pacemaker electrode arrangement according to the invention, which has a biventricular design, in the operational position,

FIG. 2 in an enlarged scale, the arrangement of a hollow puncture needle belong to the cardiac pacemaker electrode arrangement according to the invention and an insertion tube shown in their operational position before the anchoring of the cardiac pacemaker electrode,

FIG. 3 a biventricular cardiac pacemaker electrode, whose one electrode is already anchored in the myocardium, while the other is still located in the insertion tube, from which the hollow puncture needle has previously been removed, wherein a screw thread is provided as an anchor in the extension of the electrodes,

FIG. 4 a view of a biventricular cardiac pacemaker, at whose two electrodes, ring-like projections arranged one behind the other in the axial direction are provided as anchors,

FIG. 5 an embodiment modified relative to FIG. 4 with pin-like projections extending like barbs on each electrode,

FIG. 6 an embodiment modified in turn, for which a barb is provided as an anchor on the two distal ends of the electrodes,

FIG. 7 the anchoring of the distal end of an electrode belonging to an electrode arrangement according to the invention with the help of barb-like projections, which are arranged behind the distal pole in the insertion direction of the electrode,

FIG. 8 an arrangement corresponding to FIG. 7, for which pin-like projections arranged as barbs extend diagonally backwards from the electrode even directly at the distal end of the electrode, and

FIG. 9 a representation of an electrode, which is anchored in the myocardium and which has a tight screw thread as an anchor on its distal end.

### DETAILED DESCRIPTION OF THE PREFERRED EMBODIMENTS

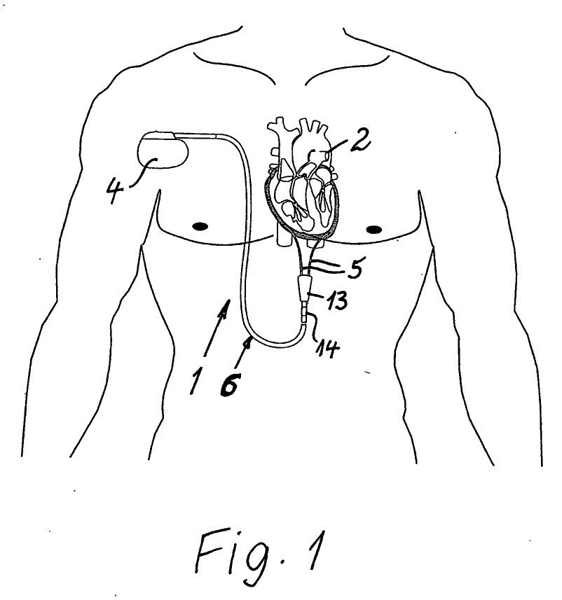

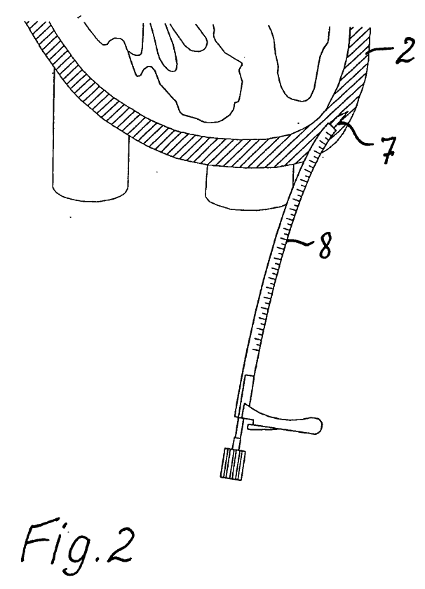

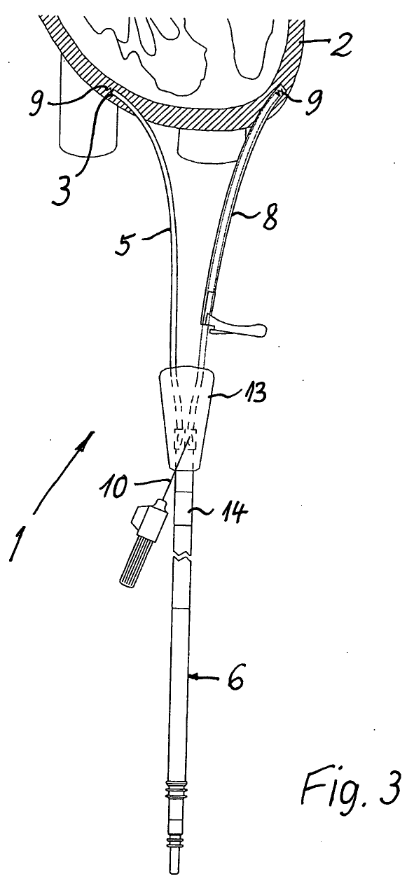

A cardiac pacemaker electrode arrangement, which is designated as a whole with 1 and which can be seen in the drawings particularly well in the common viewpoint of FIGS. 1 and 3, is provided having at least one, and in the shown embodiment, two cardiac pacemaker electrodes 5, which, in the operational position, act on the outside of a heart 2 or on the heart 2 from the outside, which are arranged with a pole 3 in the heart tissue, and which extend to an implantable cardiac pacemaker 4. Furthermore, this electrode arrangement 1 includes an electrode feed line 6 and an anchor, which is to be described in more detail below and which is shaped differently in the individual embodiments and therefore has a unique reference number for each embodiment. Each cardiac pacemaker electrode 5 or its pole 3 can be fixed with this anchor in the myocardium from outside the heart 2. Furthermore, the electrode arrangement 1 has one or more tools and/or aids, which are shown primarily in FIGS. 2 and 3 and which are explained below.

At least one hollow puncture needle 7 (FIG. 2) and one insertion tube 8 for the hollow puncture needle 7 are provided as tools or aids. Here, the inner cross section of the insertion tube 8 is dimensioned large enough that the cardiac pacemaker electrode 5 fits into this insertion tube and can move in this insertion tube with its anchor provided on the distal end, for example, a screw thread 9 explained in more detail below. One can see primarily by comparing FIGS. 2 and 3 that at first the hollow puncture needle 7 is used with the insertion tube 8, wherein the hollow needle 7 projects past the tube 8 and creates a corresponding channel in the myocardium. Then the hollow needle 7 can be withdrawn. Then, after the withdrawal of the hollow puncture needle 7, the cardiac pacemaker electrode can be inserted into the heart tissue and into the channel created therein by the hollow puncture needle 7 through the insertion tube 8 leading from the outside into the heart tissue, as can be seen clearly in FIG. 3.

The center axis of the screw thread 9 used as an anchor in this embodiment and arranged at the distal end of the cardiac pacemaker electrode 5 here continues along the longitudinal center axis of the cardiac pacemaker electrode 5, thus it is in alignment with this electrode and can have a matching or possibly even somewhat smaller or optionally also somewhat larger outer diameter than the actual cardiac pacemaker electrode 5.

Here, this cardiac pacemaker electrode 5 is flexible in a typical and known way such that it can turn or twist. There is a channel in this electrode. A piercing instrument 10, which is used as an additional tool and which fits in the region of the screw thread 9 with its working end onto a profiled section provided there, for example, a flattened shape or recess, fits into this channel, so that with this piercing instrument 10, the rotation of the screw thread 9 is possible first in a twisted position and primarily after insertion into the myocardium into a released and turned-back position, whereby the screw thread 9 and thus the cardiac pacemaker electrode 5 is anchored.

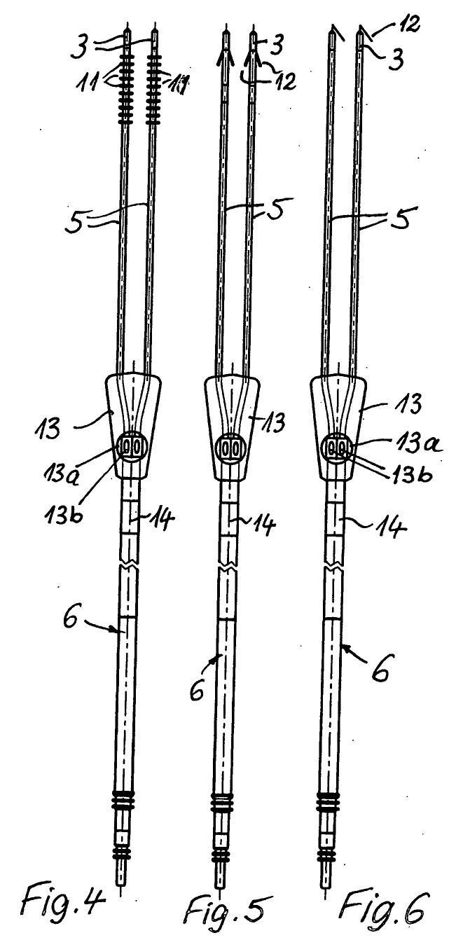

In the embodiments according to FIGS. 4 to 6, at the distal end of the cardiac pacemaker electrode 5, ring-like projections 11, optionally also umbrella-like projections or barb-shaped pin-like projections 12 are arranged as anchors, wherein these pin-like projections 12 can be provided according to FIG. 6 individually or according to FIG. 5 in pairs and here also at somewhat different positions on the corresponding cardiac pacemaker electrode 5, namely all the way at the end or at somewhat of a distance to the end. Here, the ring-like projections 11 are arranged one behind the other in the axial direction as several anchors.

In the embodiments according to FIGS. 1 and 3 to 6, the electrode arrangement 1 has a biventricular design, that is, two branching electrodes 5 that extend separately to the heart 2 are provided on a common feed line 6, which starts from the cardiac pacemaker 4. Each of these electrodes has at least one of the described anchors 9, 11, and/or 12 for attachment to the heart 2. Instead of this configuration, only one feed line 6, without a branching 13, could also be provided, which continues as an electrode 5 up to its anchor in the heart.

The branching 13 has an opening 13a for inlets 13b for the piercing instrument 10, thus it performs a double function.

In the figures, one can also see that the anode 14 of the electrode or electrodes 5 are arranged outside of the heart 2 at a distance to the cathode or to the pole 3 located on the heart and here, for a biventricular, branched electrode, in the region of the common feed line 6 in front of the branching 13, so that the actual electrodes 5 can be shaped thinner accordingly.

The cardiac pacemaker electrode arrangement 1 with a cardiac pacemaker electrode 5, in the operational position, acting on the outside of a heart 2 and arranged with a pole 3 in the heart tissue and extending to an implantable cardiac pacemaker 4 has a hollow puncture needle 7 as its associated aid and an insertion tube 8 for this hollow puncture needle 7, whose inner diameter is dimensioned large enough that, after withdrawing the hollow puncture needle 7, the cardiac pacemaker electrode 5 fits into this insertion tube 8 and can be pushed in the tube into the heart tissue. Then, the insertion tube 8 can be withdrawn in a known way or pulled out laterally through a side opening.

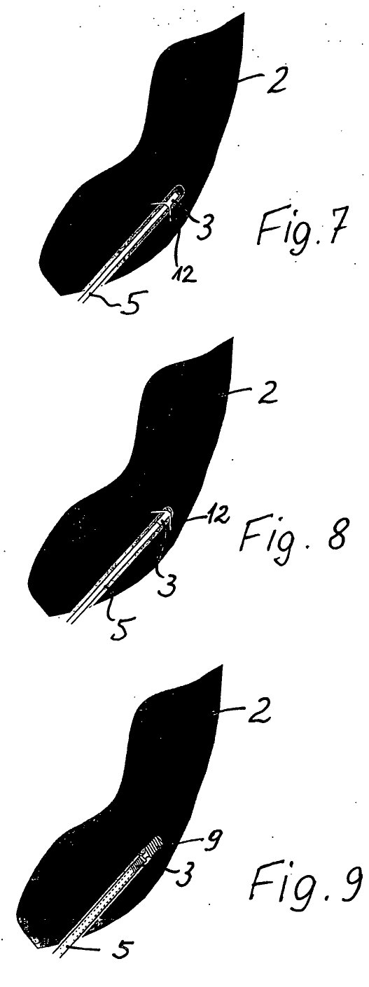

In FIGS. 7, 8, and 9, the anchored operational position of the distal end of the cardiac pacemaker electrodes 5 is also shown, which have differently shaped barb-like anchors (FIGS. 7 and 8) or a screw thread 9 as the anchor, wherein this screw thread 9 is formed according to FIG. 9 with narrow windings, which can be clamped and established within the channel located in the myocardium and created by the hollow puncture needle 7.

1. Cardiac pacemaker electrode arrangement (1) comprising a cardiac pacemaker electrode (5), which, in an operational position, acts on an outside of a heart (2) or on the heart (2) from an outside thereof and/or which is arranged or which can be fixed with a pole (3) in heart tissue and which extends to an implantable cardiac pacemaker (4) and which has an electrode feed line (6), and with an anchor that can be fixed from the outside of the heart (2) in the operational position, and at least one tool and/or aid used for positioning and/or fixing the anchor, comprising at least one hollow puncture needle (7) and one insertion tube (8) for the hollow puncture needle (7), an inner cross section of the insertion tube (8) is dimensioned large enough that the cardiac pacemaker electrode (5) fits into the tube and can move in the tube with the anchor provided on a distal end of the cardiac pacemaker electrode (5).

2. Electrode arrangement according to claim 1, wherein, after withdrawal of the hollow puncture needle (7), the cardiac pacemaker electrode (5) can be inserted into the heart tissue and into a channel formed by the hollow puncture needle (7) through the insertion tube (8) leading from the outside into the heart tissue.

3. Electrode arrangement according claim 1, wherein on the distal end of the cardiac pacemaker electrode (5) there is a screw thread (9) as the anchor, having a center axis that forms a continuation of a longitudinal center axis of the cardiac pacemaker electrode (5), the cardiac pacemaker electrode (5) is flexible, such that it can twist, and a channel is arranged therein for a piercing instrument (10) used as the tool or as an additional tool, and in a region of the screw thread (9) there is a profiled section, especially a flattened shape or recess, which fits with a working end of the piercing instrument (10) or tool with a positive fit in the direction of rotation.

4. Electrode arrangement according to claim 1, wherein at least one ring-like projection (11), umbrella-like projection, and/or barb-like projection (12) is arranged at the distal end of the cardiac pacemaker electrode (5) as the anchor or an additional anchor.

5. Electrode arrangement according claim 1, wherein several anchors (9, 11, 12) are provided one behind the other in the axial direction.

6. Electrode arrangement according to claim 1, wherein the electrode arrangement has a biventricular design and the electrode feed line (6) has two branching electrodes (5), which then run separately to the heart (2) and each of which has at least one of the anchors for attachment to the heart (2).

7. Electrode arrangement according to claim 1, wherein an anode (14) of the electrode or electrodes (5) is arranged outside of the heart (2) at a distance to a cathode or to the pole (3) located on the heart (2) and for a biventricular, branched electrode arrangement, in a region of the common feed line (6) before a branching (13) into the two electrodes.
